(19) United States
(12) Patent Application Publication
(10) Pub. No.: US 2009/0059203 A1
(43) Pub. Date: Mar. 5, 2009

(54) Apparatus For Measuring Concentration of a Specific Ingredient In-Situ

(76) Inventor: Wei-Kung Wang, Taipei (TW)

Correspondence Address: SENNIGER POWERS LLP, 100 NORTH BROADWAY, 17TH FLOOR, ST LOUIS, MO, 63102, US

(21) Appl. No.: 12/199,769

(22) Filed: Aug. 27, 2008

Related U.S. Application Data
(63) Continuation-in-part of application No. 10/123,124, filed on Apr. 16, 2002.
Application No. 10/123,124 is a continuation-in-part of application No. 09/766,237, filed on Jan. 19, 2001, now abandoned.

(30) Foreign Application Priority Data
Mar. 17, 2000 (CN) 089104938

(51) Int. Cl. G01N 33/49; A61B 5/1455

(52) U.S. Cl. 356/40; 600/322

(57) ABSTRACT

Disclosed is an apparatus for measuring the concentration of a specific ingredient in a solution. According to one embodiment of the present invention, said apparatus comprises: a signal collector for collecting a plurality of signals emitted from a target in a selected volume of the solution, and one of the signals corresponding to the selected volume; detectors for detecting the signals; and beam splitters for splitting said signals and transmitting the signals to the detectors. The present invention provides an apparatus for effectively measuring concentration in-situ without the need of extracting the solution out of its original container.

### CROSS-REFERENCE TO RELATED APPLICATION

The present application is a continuation-in-part of pending U.S. patent application Ser. No. 10/123,124, entitled “APPARATUS FOR MEASURING CONCENTRATION OF A SPECIFIC INGREDIENT IN-SITU” filed on 16 Apr. 2002, which is a continuation-in-part of a U.S. patent application Ser. No. 09/766,237, entitled “MOLD-IN METHOD AND APPARATUS” and filed on 19 Jan. 2001 by the same inventor of the present application.

### FIELD OF INVENTION

The present invention relates to an apparatus for measuring the concentration of a specific ingredient in-situ.

### BACKGROUND AND SUMMARY OF INVENTION

To measure the concentration of an ingredient in a solution is usually to have the solution extracted from its container and put into a test tube or a cuvette, which is another container with a known volume (or more precisely, a known signal path). After the specific signal generated from the specific ingredient is measured, together with the known volume, the concentration can be determined by the ratio of the amount of ingredient to the volume.

However, if such measurement is to be taken an in-situ (i.e., the solution had better not be extracted from the container such as the cases of extracting blood from the blood vessel or moving a sample out of a production line), the information about the volume is required to determine the concentration.

Therefore, for the case of measuring the concentration of one ingredient, at least two signals: one for the volume and the other for the specific ingredient, are needed for the concentration measurement. For the case of two ingredients, three signals are needed for the measurement. When there are (N−1) ingredients, by deduction, N signals including one for volume and (N−1) signals for the (N−1) ingredients are needed. In order to separate and determine each of these N signals, usually a grating is used. Then based on the ratio of the signal for each ingredient to the signal for the volume, the concentrations of N ingredient can be obtained.

For the volume signal, it can be obtained by a direct measurement of the volume by, for example, ultrasound or light reflection. Then, the length of the signal path can be determined. According to one aspect of the present invention, the specific signal from the solvent is measured, instead of measuring the volume signal. Because the solvent constitutes most of the volume in the solution, based on the signal of the solvent, the volume of the effective container can be determined even if the container does not have a well-defined shape. Besides the solvent, a marker with known concentration could also be used to determine the volume, and the signal of the marker is regarded as the volume signal. Such a marker could be either the intrinsic type or the added-in type which will be explained in detail below.

### BRIEF DESCRIPTION OF DRAWING

The present invention can be better understood through the accompanying drawing in which:

FIG. 1 shows an apparatus for measuring the concentration of a specific ingredient in-situ, according to one embodiment of the present invention.

### DETAILED DESCRIPTION OF INVENTION

In the figured embodiment, an optical signal (enamation or induced signal) is used as an example.

To further define the solution, the solution itself must have a distinct compartment which is definable. If the solvent or some solutes flow in and out of the defined compartment, the solution is not uniform and becomes difficult to define a concentration. This is usually true in tissue that the solvent of water can flow from one place to another, even between blood vessel and surrounding tissue. If there is a clear compartment like a defined container, both solvent and solute can be used as the marker for the volume. In those cases, the solvent like water is a free mover, so we need markers that can be confined in the special compartment which is definable. For examples, blood is confined in the vessels, artery, vein and capillary. Blood in artery is considered as in a compartment and every ingredient in the compartment is approximately at the same concentration, even if some minor variation may happen when small amount of water flow in and out of the large volume vessel. Similarly, blood in vein may also be considered as in one compartment.

To analyze the concentration of ingredient in these compartments, it is needed a marker for the volume which is confined within each a definable compartment, and does not migrate to the outside of the defined compartment. The volume therefore has a uniform concentration. The hemoglobin and hemoglobin related particles could be the ideal candidate markers for the volume because they can be confined within the vessel (i.e. an defined compartment). As a result, besides the method to isolate such volume as described in mode-in method and apparatus of the parent application, a new method is disclosed.

Moreover, to accurately measure both of a sample signal and the volume signal, an ingredient (or sample) as glucose and the volume signal are required to get from the same tissue. Particularly, if these signals are induced by an input signal, the input signal source(s) should be incident on the same tissue and then, the result data are collected from the targets through the collector. In the case of using an induced signal, there is a need to clamp the tissue that is to be excited. Such clamp, called “signal guide,” can be any structure that fixes the volume to be excited. The signal collector is used to fix the specific volume and time to collect signal for either enamation or induced signal.

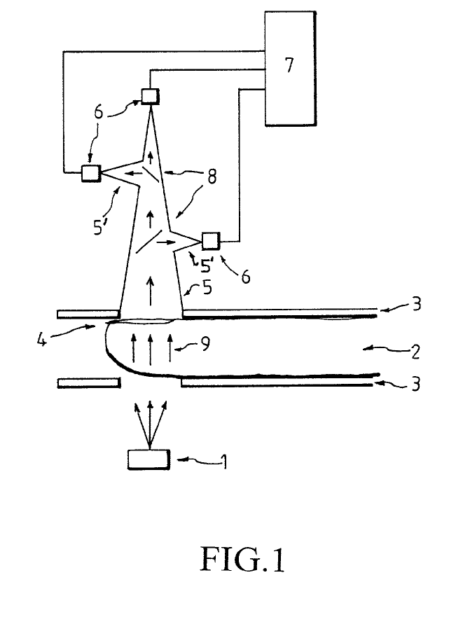

After the signals arc collected, a spectroscopic method is needed to separate these two signals and collect the signals as many as possible. A conventional way is to use grating. According to the exemplary apparatus of the present invention shown in FIG. 1, two small cones 5′ and a large cone 5 housing two dichroic beam splitters 8 are used as the signal collector to ensure a better collection of signals from the tissue.

As shown in FIG. 1, the signals are collected from the finger 2. The light from the light source 1 is incident into the inner side of the finger 2 through a signal guide (not shown in the FIGURE). After being interactive with the finger 2, the light 9 comes out from the nail 4 side of the finger 2 and is collected by the cone 5. The finger 2 is clamped by an engulfed structure such as an envelope 3 to fix the position in the finger to be investigated. Both the signal guide and collector are attached to the envelope 3, so that the signal can came from the same piece of the sample.

In order to detect the concentrations of other ingredients in the blood, other specific signals, for example, signals of uric acid, cholesterol, triglycerol oxyhemoglobin or any drugs or ingredients that are detected for their concentrations, are needed. Such signals can be detected one at a time by using the measurement apparatus shown in FIG. 1, by measuring a specific signal together with the signal of the solvent. Alternatively, several ingredients (e.g., N−1 ingredients) can be detected at the same time. In the latter case, N−1 dichroic beam splitters are needed to separate N signals, and N cones (including 1 large cone and N−1 small cones), each of which has lens to collect and focus each of the N signals into corresponding designated detectors 6. The detectors 6, which arc connected to the processing circuit 7, are set at the tips of the cones (5, 5′) so as to collect signals. A monochrometer that includes a band pass filter can be used to further refine the spectrum in each cone. The inner surfaces of the cones are made highly reflective to increase their ability to collect signals.

Instead, the signals could be enamations such as α, β or γ particles emitted from isotopes decay, or chemi-luminance-light emitted by chemical energy. The signals could also be secondary signals such as transmittance, scattering, fluorescence, Raman, etc., induced by another electromagnetic (EM) wave such as X-ray, visible, ultra-violet infrared or microwave. To generate EM wave, all kinds of laser, diode laser, light emitted diode, lamps or EM sources can be used.

For any induced signals, there is always a time delay from excitation to emission of the induced signal. The incident signal could be guided at an earlier time to excite the target in a selected volume to be measured, and after time Δt, the induced signal is collected. This method is referred to as “time resolved technique.” The technique can be used in the exemplary apparatus for reducing noise. The technique will be more useful when the exited target is moving. Assume the target is at position x with a velocity V*. After Δt, the exited target will move to x+V*Δt and emits the induced signal at this position. The target can be exited in a volume at position x, as time t, then the induced signal from the target in the specific volume is measured at x+Δx=x+V*Δt, at the time t+Δt. Thus, the noise resulted from the stationary (not moving) parts can be cut.

The signal-noise ratio can be improved by further using switches. When the switch of the guide for the input signal is on, the switch for the collector is off, when the guide for the input signal is off the switch for the collector is on. Such on-off circle can be repeated for a lot of times to improve the signal-noise ratio. The above-mentioned arrangement is very useful as the targets are moving in a conduit such as an artery or production line.

As the invention thus described, it will be obvious that the embodiments and description am not intended to limit the invention. The invention may vary in many ways. Such variations are not to be regarded as a departure from the spirit and scope of the invention, and all such modifications, as would be obvious to one skilled in the art, are intended for inclusion within the scope of the following claims.

We claim:

1. An apparatus for measuring the concentration of (N−1) ingredients in a solution in-situ, wherein N is a natural number and N>2,
said apparatus comprising:
means for selecting a volume that contains said solution:
a signal collector for collecting N signals from a target in said selected volume of the solution, one of said N signals being corresponding to said selected volume;
means for detecting said N signals;
means for separating said N signals and transmitting said N separated signals to said detecting means; and
a calculating element for obtaining a concentration of N−1 ingredients based on the ratio of the signal for each ingredient to the signal for the volume.

2. The apparatus according to claim 1, wherein said N signals comprise at least one induced signal from said selected volume in response to an input signal.

3. The apparatus according to claim 2, wherein said input signal is in the form of an electromagnetic wave.

4. The apparatus wording to claim 1, wherein said signal collector comprises a plurality of cones for collecting said signals and/or for accommodating the transmission of said signals to said detecting means.

5. The apparatus according to claim 4, wherein said detecting means comprises a plurality of detectors respectively located at the tips of said plurality of cones.

6. The apparatus according to claim 1, wherein said separating means comprises a dichroic beam splitter.

7. The apparatus according to claim 1, wherein said separating means comprises N−1 beam splitters for separating said N signals.

8. The apparatus according to claim 7, wherein said collector comprises N cones for collecting said N signals.

9. The apparatus according to claim 5, wherein each said plurality of cones comprises a lens for focusing the signal toward the corresponding detector.

10. The apparatus according to claim 4, wherein said plurality of cones comprise a highly reflective surface.

11. The apparatus according to claim 1, wherein said signal collector comprises an adapter for collecting said signals.

12. The apparatus according to claim 2, wherein said means for selecting a volume comprises a signal guide for directing said input signal into said target.

13. The apparatus according to claim 12, wherein said signal guide comprised in said means for selecting a volume directs said input signal into said target in said selected volume V at time t, and then said signal collector collects said signals from another selected volume V′, which V′ is the distribution of said target at time t=t+Δt.

14. The apparatus according to claim 13, wherein said target moves with a velocity V*, and said V′ is a linear transition from V to V+V*t.

15. The apparatus according to claim 14: wherein both said signal guide and signal collector respectively comprise a switch.

16. The apparatus according to claim 15, wherein the switch of said signal collector is open after a predetermined period of time when the switch of said signal guide is closed.

17. The apparatus according to claim 16, wherein said switches are changed between open and close for a plurality of times.

18. The apparatus according to claim 14, wherein said means for selecting a volume further comprises an envelope for securing said target.

19. The apparatus according to claim 1, wherein said signal corresponding to said selected volume is a signal corresponding to a marker confined within the compartment of said solution.

20. The apparatus according to claim 1, wherein said volume that contains said solution is a signal corresponding to a marker with known concentration comprising hemoglobin.
